(19) United States
(12) Patent Application Publication
(10) Pub. No.: US 2007/0213511 A1
(43) Pub. Date: Sep. 13, 2007

(54) Calicheamicin Conjugation by Antibody Deglycosylation

(75) Inventors: Arthur Kunz, New City, NY (US); Philip Ross Hamann, Thiells, NY (US); Justin Keith Moran, Valley Cottage, NY (US); Eugene Vidunas, Middletown, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/592,438

(22) PCT Filed: Mar. 15, 2005
(86) PCT No.: PCT/US05/08509

§ 371 Date: Sep. 14, 2006

Related U.S. Application Data
(60) Provisional application No. 60/553,112, filed on Mar. 15, 2004.

(51) Int. Cl. C07K 16/46

(52) U.S. Cl. 530/391.1

(57) ABSTRACT

The present invention provides processed for preparing a calicheamicin conjugate comprising removing glycosylation of a protein and reacting (i) an activated calicheamicin—hydrolyzable linker derivative and (ii) the deglycosylated protein. In one embodiment, the protein is an antibody, such as an anti-CD33 antibody (e.g., hp67.6), an anti-CD22 antibody (e.g., G544), an anti-Lewis Y antibody (e.g., G193), an anti-5T4 antibody (e.g., H8) or an anti-CD20 antibody (e.g., rituximab). In another embodiment, the calicheamicin derivative is an N-acyl derivative of calicheamicin or a disulfide analog of calicheamicin, such as N-acetyl gamma calicheamicin dimethyl hydrazide (N-acetyl calicheamicin DMH) and the hydrolyzable linker is 4-(4-acetylephenoxy) butanoic acid (AcBut) or (3-Acetylphenyl) acetic acid (AcPAc). Also provided are calicheamicin conjugates produced by such processes.

### FIELD OF THE INVENTION

The present invention relates to processes for the production of monomeric calicheamicin derivative/carrier conjugates having higher loading and yield with reduced low conjugate fraction (LCF) and aggregate. The invention also relates to the conjugates produced by these processes.

### BACKGROUND

The use of cytotoxic chemotherapy has improved the survival of patients suffering from various types of cancers. Used against select neoplastic diseases such as, e.g., acute lymphocytic leukemia in young people and Hodgkin lymphomas, cocktails of cytotoxic drugs can induce complete cures. Unfortunately, chemotherapy, as currently applied, does not result in complete remissions in a majority of cancers. Multiple reasons can explain this relative lack of efficacy. Among these, the low therapeutic index of most chemotherapeutics is a likely target for pharmaceutical improvement. The low therapeutic index reflects the narrow margin between the efficacious and toxic dose of a drug, which may prevent the administration of sufficiently high doses necessary to eradicate a tumor and obtain a curative effect.

One strategy to circumvent this problem is the use of a so-called magic bullet. The magic bullet consists of a cytotoxic compound that is chemically linked to an antibody. Binding a cytotoxic anticancer drug to an antibody that recognizes a tumor-associated-antigen can improve the therapeutic index of the drug. This antibody should ideally recognize a tumor-associated antigen (TAA) that is exclusively expressed at the surface of tumor cells. This strategy allows the delivery of the cytotoxic agent to the tumor site while minimizing the exposure of normal tissues. The antibody can deliver the cytotoxic agent specifically to the tumor and thereby reduce systemic toxicity.

Drug conjugates developed for systemic pharmacotherapy are target-specific cytotoxic agents. The concept involves coupling a therapeutic agent to a carrier molecule with specificity for a defined target cell population. Antibodies with high affinity for antigens are a natural choice as targeting moieties. With the availability of high affinity monoclonal antibodies, the prospects of antibody-targeting therapeutics have become promising. Toxic substances that have been conjugated to monoclonal antibodies include toxins, low-molecular-weight cytotoxic drugs, biological response modifiers, and radionuclides. Antibody-toxin conjugates are frequently termed immunotoxins, whereas immunoconjugates consisting of antibodies and low-molecular-weight drugs such as methotrexate and adriamycin are called chemoimmunoconjugates. Immunomodulators contain biological response modifiers that are known to have regulatory functions, such as lymphokines, growth factors, and complement-activating cobra venom factor (CVF). Radioimmunoconjugates consist of radioactive isotopes, which may be used as therapeutics to kill cells by their radiation or used for imaging. Antibody-mediated specific delivery of cytotoxic drugs to tumor cells is expected to not only augment their anti-tumor efficacy, but also to prevent nontargeted uptake by normal tissues, thus increasing their therapeutic indices.

Immunoconjugates using a member of the potent family of antibacterial and antitumor agents, known collectively as the calicheamicins or the LL-E33288 complex, were developed for use in the treatment of cancers. The most potent of the calicheamicins is designated γ₁^{l}, which is herein referenced simply as gamma. These compounds contain a methyltrisulfide that can be reacted with appropriate thiols to form disulfides, at the same time introducing a functional group such as a hydrazide or other functional group that is useful in attaching a calicheamicin derivative to a carrier. The calicheamicins contain an enediyne warhead that is activated by reduction of the —S—S— bond causing breaks in double-stranded DNA.

MYLOTARG®, also referred to as CMA-676 or CMA, is the only commercially available drug that works according to this principle. MYLOTARG® (gemtuzumab ozogamicin) is currently approved for the treatment of acute myeloid leukemia in elderly patients. The drug consists of an antibody against CD33 that is bound to calicheamicin by means of an acid-hydrolyzable linker. The disulfide analog of the semi-synthetic N-acetyl gamma calicheamicin was used for conjugation (U.S. Pat. Nos. 5,606,040 and 5,770,710, which are incorporated herein in their entirety). This molecule, N-acetyl gamma calicheamicin dimethyl hydrazide, is hereafter abbreviated as CM.

A number of antibody-based therapeutics are on the market, e.g. RITUXAN (rituximab) (an unlabeled chimeric human (1) specific for CD20, or are in clinical trials. These rely either on complement or ADCC-mediated killing of cells or the use of conjugated radionuclides, such as ¹³¹I or ⁹⁰Y, which have associated preparation and use problems for clinicians and patients. Although progress continues to be made in this field, most classical antitumor agents produce antibody conjugates that are relatively ineffective for a variety of reasons. Among the reasons for this ineffectiveness is the lack of potency of the chemotherapeutic agent.

The use of the monomeric calicheamicin derivative/carrier conjugates in developing therapies for a wide variety of cancers has been limited both by the availability of specific targeting agents (carriers) as well as the standard conjugation process, which results in the formation aggregates when the amount of the calicheamicin derivative that is conjugated to the carrier (i.e., the drug loading) is increased. It is desirable to have as much drug loaded on the carrier as is consistent with retaining the affinity of the carrier protein, because higher drug loading increases the inherent potency of the conjugate. The presence of aggregate, which must be removed for therapeutic applications, also makes the scale-up of these conjugates more difficult and decreases the yield of the products. The amount of calicheamicin loaded on the carrier protein (the drug loading), the amount of aggregate that is formed in the conjugation reaction, and the yield of final purified monomeric conjugate that can be obtained are all related. A compromise must therefore be made between higher drug loading and the yield of the final monomer by adjusting the amount of the reactive calicheamicin derivative that is added to the conjugation reaction.

The tendency for calicheamicin conjugates to aggregate is especially problematic when the conjugation reactions are performed with the linkers described in U.S. Pat. No. 5,877,296 and U.S. Pat. No. 5,773,001, which are incorporated in their entirety. In this case, a large percentage of the conjugates produced are in an aggregated form, and it is quite difficult to purify conjugates made by these original procedures for therapeutic administration. For some carrier proteins, conjugates with even modest loadings are virtually impossible to make except on small scale. Consequently, there is a need for methods for conjugating cytotoxic drugs such as the calicheamicins to carriers which minimize that amount of aggregation and thereby allow for as high a drug loading as possible with a reasonable yield of product.

Previously, a conjugation method for preparing monomeric calicheamicin derivative/carrier with higher drug loading/yield and decreased aggregation was disclosed (see U.S. Pat. Nos. 5,714,586 and 5,712,374, incorporated herein in their entirety). Although this process decreased aggregation substantially, it was discovered later that it produced conjugates containing undesirably high levels (50-60%) of a low conjugated fraction (LCF) (a fraction consisting mostly of unconjugated antibody and smaller amounts of conjugate with 1 or 2 molecules of calicheamicin/molecule of protein). The presence of the LCF in the conjugate is undesirable because it wastes the antibody, and also because it would compete with the more highly conjugated calicheamicin-carrier conjugate for the target and consequently reduce its efficacy. Therefore, an improved conjugation process that would result in significantly lower levels of the LCF, but with acceptable levels of aggregation without significantly altering the physical properties of the molecule is desirable.

### SUMMARY OF THE INVENTION

The present invention provides processed for preparing a calicheamicin conjugate comprising removing glycosylation of a protein and reacting (i) an activated calicheamicin—hydrolyzable linker derivative and (ii) the deglycosylated protein. In one embodiment, the protein is an antibody, such as an anti-CD33 antibody (e.g., hp67.6), an anti-CD22 antibody (e.g., G544), an anti-Lewis Y antibody (e.g., G193), an anti-5T4 antibody (e.g., H8) or an anti-CD20 antibody (e.g., rituximab). In another embodiment, the calicheamicin derivative is an N-acyl derivative of calicheamicin or a disulfide analog of calicheamicin, such as N-acetyl gamma calicheamicin dimethyl hydrazide (N-acetyl calicheamicin DMH) and the hydrolyzable linker is 4-(4-acetylephenoxy) butanoic acid (AcBut) or (3-Acetylphenyl) acetic acid (AcPAc). Also provided are calicheamicin conjugates produced by such processes.

### BRIEF DESCRIPTION OF THE DRAWINGS

FIG. 1 is a graph of tumor size versus time (days) of RL tumor cell xenografts following treatment with conjugates of the humanized 544 antibody, both native and deglycosylated.

FIG. 2 shows the structure of the G193 oligosaccharide and resulting endoglycosidase activity.

### DETAILED DESCRIPTION OF THE INVENTION

It is possible to improve the efficiency of conjugation of cytotoxic molecules such as calicheamicin to proteins by taking into account the protein's glycosylation, and either altering or masking its effects on conjugation. It has been shown that the presence of oligosaccharides attached to antibody or other protein molecules adversely affects the conjugation of calicheamicin to the protein, leading to the formation of protein aggregates. This may be due to the overloading of calicheamicin on only a fraction of the protein that does not have complete glycosylation, or it may have its cause in the acceleration in the conjugation rate upon the addition of one or two calicheamicins to the protein. Upon removal of all or portions of the oligosaccharides by enzymatic digestion (deglycosylation) the protein is capable of conjugating more calicheamicin on average without the adverse formation of aggregated protein. It has been shown the conjugate derived from deglycosylated protein has a more uniform loading distribution than native antibody conjugates, and that it has unaltered biological activity, except for the advantages arising from higher/more uniform conjugation. Thus, the present application is directed to a process for producing cytotoxic agent/carrier conjugates having higher loading and yield with reduced low conjugate fraction (LCF) and aggregate.

The presence of oligosaccharide on antibody may act as a hindrance in the conjugation of the therapeutic agent calicheamicin. Use of oligosaccharide deficient proteins by removal of sugar residues or the prevention of their attachment leads to a protein that is more easily and efficiently conjugated. Separation of the different subspecies of glycoprotein based on oligosaccharide content can be used to benefit the calicheamicin conjugation reaction. Any suitable method can be used to separate the different subspecies of glyoproteins and to determine oligosaccharide removal, examples of which include polyacrylamide gel electrophoresis (PAGE), size exclusion chromatography(SEC), isoelectric focusing(IEF), mass spectroscopy(MS), and ConA binding analyses

Also, there may be a beneficial effect of producing non-glycosylated protein by genetically engineering the protein to remove the glycosylation recognition sequences from proteins to prevent glycosylation. A method for achieving a preferred glycosylation pattern involves the manipulation of glycosylation either via regulatory enzymes through the addition or activation of inhibitors, or by other means such as but not limited to site directed mutagenesis, cell culturing conditions, and selection of glycosylation deficient clones.

Similar effects of glycosylation on conjugation may also apply to additional proteins and conjugated species.

As discussed previously, calicheamicin refers to a family of antibacterial and antitumor agents, as described in U.S. Pat. No.4,970,198 (see also U.S. Pat. No. 5,108,912, both of which are herein incorporated in their entirety). In one preferred embodiment of the present process, the calicheamicin is an N-acyl derivative of calicheamicin or a disulfide analog of calicheamicin. The dihydro derivatives of these compounds are described in U.S. Pat. No. 5,037,651 and the N-acylated derivatives are described in U.S. Pat. No. 5,079,233, both patents are incorporated in their entirety herein. Related compounds, which are also useful in this invention, include the esperamicins, described in U.S. Pat. Nos. 4,675,187; 4,539,203; 4,554,162; and 4,837,206, all of which are incorporated in their entirety herein. All of these compounds contain a methyltrisulfide that can be reacted with appropriate thiols to form disulfides, at the same time introducing a functional group such as a hydrazide or similar nucleophile. Two compounds that are useful in the present invention are disclosed in U.S. Pat. No. 5,053,394, and are shown in Table 1 of U.S. Pat. No. 5,877,296, gamma dimethyl hydrazide and N-acetyl gamma dimethyl hydrazide. All information in the above-mentioned patent citations is incorporated herein by reference.

Preferably, in the context of the present invention, the calicheamicin is N-acetyl gamma calicheamicin dimethyl hydrazide (N-acetyl calicheamicin DMH). N-acetyl calicheamicin DMH is at least 10- to 100-fold more potent than the majority of cytotoxic chemotherapeutic agents in current use. Its high potency makes it an ideal candidate for antibody-targeted therapy, thereby maximizing antitumor activity while reducing nonspecific exposure of normal organs and tissues.

Thus, in one embodiment, the conjugates of the present invention have the formula:
Pr(—X—W)ₘ
wherein:
Pr is a deglycosylated protein, preferably an antibody;
X is a linker that comprises a product of any reactive group that can react with the antibody;
W is a cytotoxic drug from the calicheamicin family;
m is the average loading for a purified conjugation product such that the calicheamicin constitutes 3-9% of the conjugate by weight; and
(—X—W)ₘ is a cytotoxic drug derivative
Preferably, X has the formula
   (CO-Alk¹-Sp¹-Ar- Sp²-Alk²-C(Z¹)=Q-Sp)
   wherein
Alk and Alk² are independently a bond or branched or unbranched (C₁-C₁₀) alkylene chain;
Sp¹ is a bond, —S—, —O—, —CONH—, —NHCO—, —NR—, —N(CH₂CH₂)₂N—, or —X—Ar—Y—(CH₂)ₙ—Z wherein X, Y, and Z are independently a bond, —NR—, —S—, or —O—, with the proviso that when n=0, then at least one of Y and Z must be a bond and Ar is 1,2-, 1,3-, or 1,4-phenylene optionally substituted with one, two, or three groups of (C₁-C₅) alkyl, (C₁-C₄) alkoxy, (C₁-C₄) thioalkoxy, halogen, nitro, —COOR, —CONHR, —(CH₂)ₙCOOR, —S(CH₂)ₙCOOR, —O(CH₂)ᵣCONHR, or —S(CH₂)ₙCONHR, with the proviso that when Alk¹ is a bond, Sp¹ is a bond;
n is an integer from 0 to 5;
R is a branched or unbranched (C₁-C₅) chain optionally substituted by one or two groups of —OH, (C₁-C₄) alkoxy, (C₁-C₄) thioalkoxy, halogen, nitro, (C₁-C₃) dialkylamino, or (C₁-C₃) trialkylammonium -A⁻ where A⁻ is a pharmaceutically acceptable anion completing a salt;
Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR wherein n and R are as hereinbefore defined or a 1,2-, 1,3-, 1,4-, 1,5-, 1,6-, 1,7-, 1,8-, 2,3-, 2,6-, or 2,7-naphthylidene or 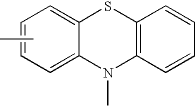
with each naphthylidene or phenothiazine optionally substituted with one, two, three, or four groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, or —S(CH₂)ₙCONHR wherein n and R are as defined above, with the proviso that when Ar is phenothiazine, Sp¹ is a bond only connected to nitrogen;
Sp² is a bond, —S—, or —O—, with the proviso that when Alk² is a bond, Sp² is a bond;
Z¹ is H, (C₁-C₅) alkyl, or phenyl optionally substituted with one, two, or three groups of (C₁-C₅) alkyl, (C₁-C₅) alkoxy, (C₁-C₄) thioalkoxy, halogen, nitro, —COOR, —ONHR, —O(CH₂)ₙCOOR, —S(CH₂)ₙCOOR, —O(CH₂)ₙCONHR, or —S(CH₂)ₙCONHR wherein n and R are as defined above;
Sp is a straight or branched-chain divalent or trivalent (C₁-C₁₈) radical, divalent or trivalent aryl or heteroaryl radical, divalent or trivalent (C₃-C₁₈) cycloalkyl or heterocycloalkyl radical, divalent or trivalent aryl- or heteroaryl-aryl (C₁-C₁₈) radical, divalent or trivalent cycloalkyl- or heterocycloalkyl-alkyl (C₁-C₁₈) radical or divalent or trivalent (C₂-C₁₈) unsaturated alkyl radical, wherein heteroaryl is preferably furyl, thienyl, N-methylpyrrolyl, pyridinyl, N-methylimidazolyl, oxazolyl, pyrimidinyl, quinolyl, isoquinolyl, N-methylcarbazoyl, aminocourmarinyl, or phenazinyl and wherein if Sp is a trivalent radical, Sp can be additionally substituted by lower (C₁-C₅) dialkylamino, lower (C₁-C₅) alkoxy, hydroxy, or lower (C₁-C₅) alkylthio groups; and
   Q is ═NHNCO—, ═NHNCS—, ═NHNCONH—, ═NHNCSNH—, or ═NHO—.
Preferably, Alk¹ is a branched or unbranched (C₁-C₁₀) alkylene chain; Sp is a bond, —S—, —O—, —CONH—, —NHCO—, or —NR wherein R is as hereinbefore defined, with the proviso that when Alk¹ is a bond, Sp¹ is a bond;
Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR wherein n and R are as hereinbefore defined, or Ar is a 1,2-, 1,3-, 1,4-, 1,5-, 1,6-, 1,7-, 1,8-, 2,3-, 2,6-, or 2,7-naphthylidene each optionally substituted with one, two, three, or four groups of (C₁-C₆) alkyl, (C₁-C₅) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or —S(CH₂)ₙCONHR.
Z¹ is (C₁-C₅) alkyl, or phenyl optionally substituted with one, two, or three groups of (C₁-C₅) alkyl, (C₁-C₄) alkoxy, (C₁-C₄) thioalkoxy, halogen, nitro, —COOR, —CONHR, —O(CH₂)ₙCOOR, —S(CH₂)ₙCOOR, —O(CH₂)ₙCONHR, or -S(CH₂)ₙCONHR.
Alk² and Sp² are together a bond.
Sp and Q are as immediately defined above.

The conjugates of the present invention utilize the cytotoxic drug calicheamicin derivatized with a linker that includes any reactive group which reacts with an antibody, which is used as a proteinaceous carrier targeting agent to form a cytotoxic drug derivative-antibody conjugate. U.S. Pat. Nos. 5,773,001; 5,739,116 and 5,877,296, incorporated herein in their entirety, discloses linkers that can be used with nucleophilic derivatives, particularly hydrazides and related nucleophiles, prepared from the calicheamicins. These linkers are especially useful in those cases where better activity is obtained when the linkage formed between the drug and the linker is hydrolyzable. These linkers contain two functional groups. One group typically is a carboxylic acid that is utilized to react with the carrier. The acid functional group, when properly activated, can form an amide linkage with a free amine group of the carrier, such as, for example, the amine in the side chain of a lysine of an antibody or other proteinaceous carrier. The other functional group commonly is a carbonyl group, i.e., an aldehyde or a ketone, which will react with the appropriately modified therapeutic agent. The carbonyl groups can react with a hydrazide group on the drug to form a hydrazone linkage. This linkage is hydrolyzable, allowing for release of the therapeutic agent from the conjugate after binding to the target cells.

Preferably, the hydrolyzable linker is 4-(4-acetylphenoxy) butanoic acid (AcBut) or (3-Acetylphenyl) acetic acid (AcPAc). In addition to the benefit of deglycosylation seen for the hydrolyzable hybrid linkers AcBut and AcPAc, a similar improvement on loading and aggregate reduction is seen with the non-hydrolyzable DMAmide linkage (also referred to as DM derivatives of calicheamicin), indicating a universal effect for all the calicheamicin derivatives.

N-hydroxysuccinimide (OSu) esters or other comparably activated esters can be used to generate the activated calicheamicin—hydrolyzable linker derivative. Examples of other suitable activating esters include NHS (N-hydroxysuccinimide), sulfo-NHS (sulfonated NHS), PFP (pentafluorophenyl), TFP (tetrafluorophenyl), and DNP (dinitrophenyl).

The present invention relates to conjugation of calicheamicin to any protein. The main determinant of protein structure, and therefore function, is its primary amino acid sequence, which is produced by translation at the ribosome. Many proteins, including antibodies or immunoglobulins, however, undergo posttranslational modifications in the cells in which they are produced. These modifications can include the attachment of many different moieties including methyl, acetyl, phosphoryl groups, nucleic acids, lipids or carbohydrates. Attachment of carbohydrates (glycosides) leads to the formation of a glycoprotein in the process known as glycosylation. A large number of proteins are found to be glycosylated and the sugar groups have been shown to be linked either through the protein serine/threonine(O-linked) or asparagine(N-linked) residues. Thus, any suitable protein having modifications, preferably glycosylation, can be conjugated according to the present invention.

A majority of the recombinant proteins currently being produced or being evaluated for use as therapeutic agents exist as glycoproteins. Glycosylation can play an important role in the function of a protein, as seen with tissue plasminogen activator, or may not be essential for activity, as seen with gamma interferon. Aside from effects on biological activity, glycosylation can also affect the proteins physicochemical properties such as solubility, mass, and ionic charge. Thus, the present methods take advantage of this.

In the case of antibodies or immunoglobulins (Ig), glycosylation is accomplished via an N-linkage that preferentially occurs on the Fc portion of the molecule in the heavy chain constant region's second domain (CH2). The glycosylation of antibodies is usually seen as a complex biantennary structure. Crystallographic analysis of the Ig Fc has shown that the oligosaccharide attached at this position is sequestered internal to the two CH2 heavy chain domains. The attachment of sugars on the heavy chain constant domain does not appear to have a major role in determining the affinity and /or specificity of the antibody molecule, but is essential for effector functions such as complement fixation, and may affect pharmacokinetic properties. In a minority of cases, on the variable region domains, either heavy or light chains, it may have a greater impact on antigen binding affinity and specificity.

Regardless of the region of the molecule on which the sugars are attached the linkage occurs at a consensus amino-acid sequence of Asn-XXX-Ser/Thr and the attachment is mediated by an enzymatic process involving glycosyltransferases within the cell. By weight, the sugar moieties of the oligosaccharide on Ig G accounts for about 2-3% of the mass of approximately 150,000 D. The glycosylation of Ig can be asymmetric or truncated thus creating a heterogeneous distribution of glycoforms having various oligosaccharide chain lengths. Such heterogeneity can be exhibited by techniques such as isoelectric focusing or more clearly by oligosaccharide analyses. The type and distribution of these glycoforms is dependent upon the protein expression system being used for producing the protein, conditions under which cells are grown, clonal heterogeneity of cell lines, as well as extracellular processes such as deamidation.

Examples of antibodies that may be used in the present invention include monoclonal antibodies (mAbs), for example, chimeric antibodies, humanized antibodies, primatized antibodies, resurfaced antibodies, human antibodies and biologically active fragments thereof. The term antibody, as used herein, unless indicated otherwise, is used broadly to refer to both antibody molecules and a variety of antibody derived molecules. Such antibody-derived molecules comprise at least one variable region (either a heavy chain or light chain variable region) and include molecules such as Fab fragments, F(ab′)₂ fragments, Fd fragments, Fabc fragments, Sc antibodies (single chain antibodies), diabodies, individual antibody light single chains, individual antibody heavy chains, chimeric fusions between antibody chains and other molecules, and the like.

The antibodies of the present invention can be specific for any antigen and, preferably, are specific for a TAA. Example of suitable antigens include CD22, CD33, HER2/neu; EGFR; PSMA; PSCA; MIRACL-26457; CEA; Lewis Y (Le^{y}) and 5T4. Exemplary antibodies include hp67.6 and g5/44, which are humanized IgG4 antibodies that specifically recognize human CD33 or CD22, respectively (see U.S. Pat. No. 5,773,001 and U.S Application Nos. 2004/0082764 A1 and 2004/0192900 A1, which are incorporated herein in their entirety). RITUXAN (IDEC Pharmaceuticals Corporation and Genentech), which is a chimeric IgG1-k antibody that recognizes CD20, is also an exemplary antibody. Another example is an anti-Lewis Y antibody designated hu3S193 (see U.S. Pat. Nos. 6,310,185; 6,518,415; 5,874,060, which are incorporated herein in their entirety) or, alternatively, G193, which is described in co-pending application entitled “Calicheamicin Conjugates” (AM101462). One additional exemplary antibody is the humanized H8 antibody, which is specific for 5T4.

The antibodies of the subject invention may be produced by a variety of methods useful for the production of polypeptides, e.g., in vitro synthesis, recombinant DNA production, and the like. Preferably, the antibodies are produced by recombinant DNA technology and protein expression methods. Techniques for manipulating DNA (e.g., polynucleotides) are well known to the person of ordinary skill in the art of molecular biology. Examples of such well-known techniques can be found in Molecular Cloning: A Laboratory Manual 2^{nd} Edition, Sambrook et al, Cold Spring Harbor, N.Y. (1989). Techniques for the recombinant expression of immunoglobulins, including humanized immunoglobulins, can also be found, among other places in Goeddel et al, Gene Expression Technology Methods in Enzymology, Vol. 185, Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993). Examples of conventional molecular biology techniques include, but are not limited to, in vitro ligation, restriction endonuclease digestion, PCR, cellular transformation, hybridization, electrophoresis, DNA sequencing, and the like.

The general methods for construction of vectors, transfection of cells to produce host cells, culture of cells to produce antibodies are all conventional molecular biology methods. Likewise, once produced, the recombinant antibodies can be purified by standard procedures of the art, including cross-flow filtration, ammonium sulphate precipitation, affinity column chromatography, gel electrophoresis, diafiltration and the like. The host cells used to express the recombinant antibody may be either a bacterial cell, such as E. coli, or preferably, a eukaryotic cell. Preferably, a mammalian cell such as a PER.C.6 cell or a Chinese hamster ovary cell (CHO) is used. The choice of expression vector is dependent upon the choice of host cell, and is selected so as to have the desired expression and regulatory characteristics in the selected host cell.

In the context of the present invention, a monomeric cytotoxic drug conjugate refers to a single antibody covalently attached to any number of calicheamicin molecules without significant aggregation of the antibodies. The number of calicheamicin moieties covalently attached to an antibody is also referred to as drug loading. For example, according to the present invention, the average loading can be anywhere from 0.1 to 10 or 15 calicheamicin moieties per antibody. A given population of conjugates (e.g., in a composition or formulation) can be either heterogeous or homogenous in terms of drug loading. In a heterogenous population, since average loading represents the average number of drug molecules (or moles) conjugated to an antibody, the actual number of drug moieties per antibody can vary substantially. The percentage of antibody in a given population having unconjugated or significantly under-conjugated antibody is referred to as the low conjugate fraction or LCF. Preferably, the LCF of proteins conjugated using the present invention is less that 10%, more preferably, less than 5% and, most preferably, less than 2.5%.

When conjugating the unmodified antibodies, it is necessary to add excipients to the reaction mixture in order to get adequate incorporation of the calicheamicin with acceptable yield and levels of aggregate. These excipients, in addition to helping to solubilize the calicheamicin, may also produce a masking effect on the oligosaccharide normally interfering with conjugation. Upon deglycosylation, as mentioned previously, conjugation can be run without the use of excipients. For example, the hP67.6 antibody was conjugated without the use of excipients, which resulted in acceptable calicheamicin loading of 4-5 M/M with high yield and low aggregate.

Use of particular excipients, e.g., cosolvents, additives, and specific reaction conditions together with the separation process can result in the formation of a monomeric cytotoxic drug derivative antibody conjugate with even more significant reduction in the LCF. The monomeric form of the conjugates as opposed to the aggregated form has significant therapeutic value, and minimizing the LCF and substantially reducing aggregation results in the utilization of the antibody starting material in a therapeutically meaningful manner by preventing the LCF from competing with the more highly conjugated fraction (HCF). General or typical reaction conditions have been described previously and can be found, for example, in U.S. Pat. Nos. 5,773,001; 5,739,116; and 5,877,296; U.S Application Nos. 2004/0082764 A1 and 2004/0192900 A1; and International Application No. WO 03/092623, all of which are incorporated herein in their entirety.

The amount of excipient necessary to effectively form a monomeric conjugate also varies from antibody to antibody. This amount can also be determined by one of ordinary skill in the art without undue experimentation. The reactions can be performed in compatible buffer at a pH of about 7 to about 9, at a temperature ranging from about 25° C. to about 40° C., and for times ranging from 15 minutes to 24 hours. Those who are skilled in the art can readily determine acceptable pH ranges for other types of conjugates. For various antibodies the use of slight variations in the combinations of the aforementioned additives have been found to improve drug loading and monomeric conjugate yield, and it is understood that any particular antibody may require some minor alterations in the exact conditions or choice of additives to achieve the optimum results.

Following conjugation, the monomeric conjugates may be purified from unconjugated reactants (such as proteinaceous carrier molecules/antibodies and free cytotoxic drug/calicheamicin) and/or aggregated form of the conjugates. Conventional methods for purification, for example, size exclusion chromatography (SEC), hydrophobic interaction chromatography (HIC), ion exchange chromatography (IEC), chromatofocusing (CF), can be used. Following, for example, chromatographic separation, the conjugate can be ultrafiltered and/or diafiltered.

All references and patents cited above are incorporated herein by reference. Numerous modifications and variations of the present inventions are included in the above-identified specification and are obvious to one of skill in the art and are encompassed within the scope of the claims.

### EXAMPLES

### Example 1

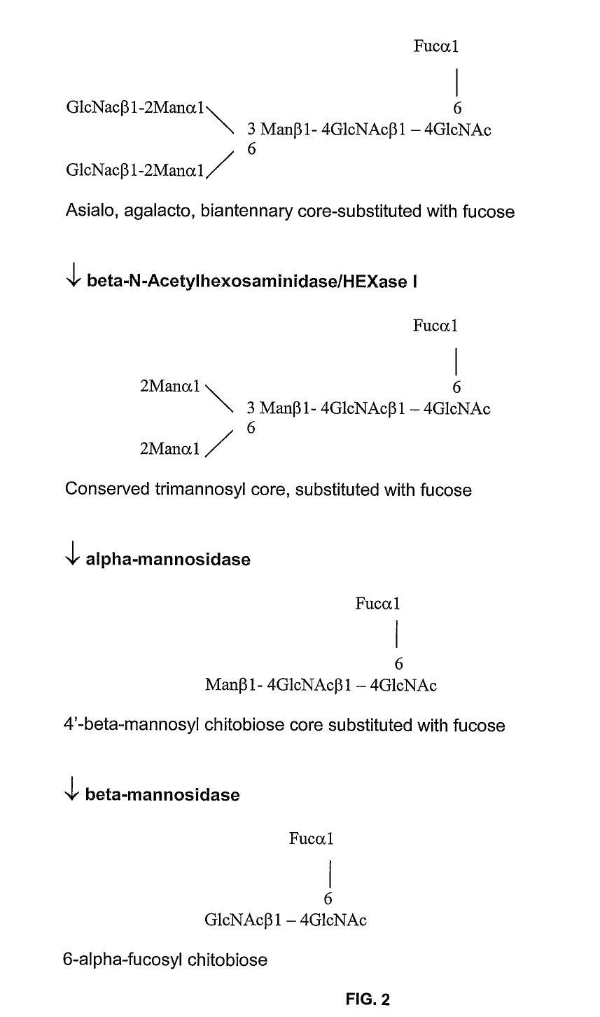

The present example demonstrates that the percent aggregate decreased with each stepwise monosaccharide removal from the G193 oligosaccharide. In contrast, the percent unconjugated protein decreased only when the 6-alpha-fucosyl chitobiose (after treatment with b-mannose) fragment was left attached to the protein. This is shown below in Table 1 (Percent Aggregate and Percent Free Protein from glycosidase experiments). FIG. 2 shows the resulting cleavages of the G193 antibody.

All conjugation reactions were performed using the same reaction conditions, with the exception of the enzymatically treated antibody. The final reaction conditions were as follows: 200 mM octanoate, 50 mM HEPBS, 5% (w/w) calicheamicin, 5 mg/mL antibody, pH 8.2. The solutions were incubated at 32° C. for one hour. For example, a typical 1 mL conjugation reaction using the G193 antibody and sodium octanoate as the additive was performed as follows. To a glass reaction vial containing a magnetic stir bar, 0.334 mL (10 mg) of G193 and 0.451 mL of water were added. To this solution, 0.05 mL of 1 M HEPBS pH 8.56 was added to give a final concentration of 50 mM. Next 0.10 mL of 2M sodium octanoate was added to give a final concentration of 200 mM, and the solution was well mixed. The pH was measured and no further adjustments were made if it was 8.3±0.2. As the solution was stirring, 0.065 mL of 10.7 mg/mL CL-191548 was added to a final concentration of 7% (w/w of mAb). The solution was then placed in a 32±2° C. bath for one hour. After one hour the solution was removed from the bath and allowed to cool to room temperature. The solution was transferred to a 1.5 mL eppendorf tube and centrifuged at 14000 rpm for two minutes. The solution was then analyzed for aggregate and unconjugated protein. For the other additives, the final concentrations were 37.5 mM (sodium decanoate) and 10 mM (sodium deoxycholate).

Beta-N-Acetyl Hexosaminadase (Prozyme, San Leandro, Calif.) was used to cleave the two terminal GlcNAc s from the glycan. 400 μL of the 5× buffer (Prozyme) was added to 600 μL water to produce a 2× buffer. 40 μL p-N-Acetyl Hexosaminadase was added to the 2× buffer. Ten μL of this enzyme solution was then added to 1000 μL of 30 mg/mL G193. The reaction solution was purified on a Superdex 200HR column (10/30) using 10 mM citrate/75 mM NaCl pH 5.0. Pooled fractions were concentrated and used for subsequent reactions.

Alpha-Mannosidase (Sigma) was used to cleave the Manα-6 and Manα-3. The buffer used was 0.25M sodium acetate pH 4.5. Eighty-three μL of the α-Mannosidase was added to 1.5 mL of the 17 mg/ml p-N-Acetyl Hexosaminadase treated G193 solution (GI 93-hex). 40 μL buffer and 17 μL water were then added. The reaction solution was purified on a Superdex 200HR column (10/30) using 10 mM citrate/75 mM NaCl pH 5.0. Pooled fractions were concentrated and used for subsequent reactions.

Beta-Mannosidase (Sigma) was used to cleave the p-Mannose from the glycan. Forty μL of β-Mannosidase was added to 500 μL of the 25 mg/ml α-Mannosidase treated G193-hexsolution (G193-hex-manox). The reaction solution was purified on a Superdex 200HR column (10/30) using 10 mM citrate/75 mM NaCl pH 5.0. Pooled fractions were concentrated and used for subsequent reactions.

After the reaction solution was centrifuged, a 0.02 mL sample was removed and added to 0.180 mL of 50 mM HEPBS pH 8.5. This solution was transferred to a HPLC vial and 0.10 mL was injected on BioRad Bio-Sil SEC250 column using 0.020M Tris/0.10M NaCl as the eluant with an isocratic elution, and a flow rate of 1.0 mL/min. The absorbance was monitored at 280 nm. The percent aggregate is reported as the area percent (A280) and was not corrected for calicheamicin contribution.

After the reaction solution was centrifuged, a 0.020 mL sample was removed and added to 0.180 mL of 0.66M potassium phosphate pH 8.6. This solution was transferred to a HPLC vial and 0.10 mL was injected on a Tosoh Butyl NPR column using a gradient elution of 0 to 100% B over 20 min at a flow rate of 1 mL/min. The absorbance was monitored at 280 nm. The percent unconjugated protein is reported as the area percent (A280) and was not corrected for calicheamicin contribution.

After the reaction solution was centrifuged, a 0.025 mL sample was removed and added to 0.475 mL of 50 mM HEPBS pH 8.5. The solution was analyzed at 280 nm, 310 nm and 390 nm in a Shimatzu spectrophotometer. The protein concentration and drug loading were calculated using the CMD-193 equation.

TABLE 1

| | Percent | | |
|---|---|---|---|
| | Unconjugated | Percent | Drug Loading |
| mAb | Protein | Aggregate | (mcg/mg) |
| G193 | 15.7 | 38.27 | 47.94 |
| G193-hex | 16.02 | 24.88 | 44.27 |
| G193-hex-alpha | 13.98 | 16.65 | 49.71 |
| G193-hex-alpha-beta | 3.8 | 10.62 | 83.74 |

Protein (mg/mL) = [(A280-A390) − 1.62(A310-A390)]/1.33
Calicheamicin (mcg/mL) = [1000(A310-A390)]/11.21
Drug Loading (mcg/mg) = Calicheamicin/Protein

### Example 2

The present example shows the effects of deglycosylation on the hP67.6 antibody (anti-CD33 antibody).

To determine the effects of deglycosylation, antibody hP67.6 was deglycosylated using endoglycosidase F (peptide-N-glycosidase F), which cleaves N linked oligosaccharide at its peptide linkage, as follows. The antibody was adjusted to pH 7.5 with PBS and incubated at 37° C. under non-denaturing conditions overnight with enzyme at a ratio of 2 units enzyme per 10 mg antibody. The enzyme treated antibody was then purified by size exclusion chromatography, and then concentrated in PBS. Confirmation of oligosaccharide removal was seen by polyacrylamide gel electrophoresis (PAGE), size exclusion chromatography(SEC), isoelectric focusing(IEF), mass spectroscopy(MS), and ConA binding analyses.

Using typical conjugation conditions, such as those described above in Example 1, conjugation of the deglycosylated hP67.6 antibody with the N-acetyl gamma DMH AcBut OSu derivative of calicheamicin resulted in a monomeric conjugate with a calicheamicin loading of 12 moles/mole antibody with less than 10% aggregated protein present. A partially deglycosylated preparation of the hP67.6 antibody resulted in a calicheamicin loading of approximately 7-8 moles/mole antibody also with less than 10% aggregated protein. Conjugation of the untreated antibody under similar conditions resulted in a monomeric conjugate fraction with a calicheamicin loading of only 3-4 moles/mole antibody.

Glycosidase treatment of a non-glycosylated protein, human serum albumin (HSA) had no affect on the calicheamicin conjugation of the protein.

### Example 3

An optimized method for conjugating the N-acetyl gamma calicheamicin DMH AcBut OSu (AcBut) derivative to hP67.6 antibody was used to produce a batch of conjugate (as described above in Example 1, for example). This process yielded approximately 75%, by protein weight added, monomeric conjugate with an average calicheamicin loading of approximately 3 M/M and less than 5% total aggregated protein and unbound calicheamicin.

Using the same hP67.6 antibody lot which was deglycosylated as described above in Example 2 and the same conjugation conditions, the yielded was 100%, by protein weight added, monomeric conjugate having an average calicheamicin loading of 5M/M with less than 2% total aggregate and unbound calicheamicin.

### Example 4

Antibody gCTM01, which has the same human IgG4 Fc construct as the hP67.6 antibody (anti-CD33 antibody), was deglycosylated similarly to the conditions described above in Example 2. This deglycosylated antibody was conjugated using, for example, typical conditions described above in Example 1, to give a calicheamicin loading of over 6 M/M antibody with an almost 100% yield of monomeric conjugate with 1-2% aggregate. Similar conjugation of the unmodified antibody under the same conditions resulted in a calicheamicin loading of only 3-4 M/M antibody and a yield of monomeric conjugate in the 50-75% range with aggregate ranging from 5-10%.

### Example 5

The humanized antibody A33 (antibody specific for the A33 antigen, which is a glycoprotein homogeneously expressed by >95% of human colon cancers and by normal colon cells) was seen to conjugate similar to gCTM01 using conjugation conditions similar to those shown above in Example 1. Deglycosylation and subsequent conjugation of this antibody with the calicheamicin AcBut derivative had results similar to the deglycosylated gCTM01 antibody (as described above) in which the loading was greater than 6 M/M, with 1-2% aggregate and almost 100% yield of protein.

### Example 6

Neuraminidase treatment of the hP67.6 antibody failed to give the improvement in conjugation (using typical reactions conditions such as those described above in Example 1) seen upon complete deglycosylation. This indicated that the effect seen was not due to simple removal of the terminal sialic acid on the antibody oligosaccharide.

### Example 7

Under the best conjugation conditions found for the Herceptin antibody (anti-her2/neu antibody), monomeric conjugate was obtained with a calicheamicin loading of approximately 3M/M antibody containing less than 5% aggregate with an approximately 50% yield (by protein weight). When deglycosylated using the conditions described above in Example 2, when the antibody was conjugated under similar conditions, the result was a monomeric conjugate with a loading of 8M/M containing less than 5% aggregate and with a yield greater than 90%.

### Example 8

Under the best conjugation conditions found for the Rituxumab antibody (the C2B8 antibody, an anti-CD22 antibody), monomeric conjugate was obstained with a calicheamicin loading of 2-3 M/M antibody with less than 5% aggregate and approximately 50% yield. When deglycosylated using the conditions described above in Example 2, the antibody conjugated with a calicheamicin loading greater than 7M/M antibody with less than 5% aggregate and a yield of approximately 80%.

### Example 9

CTLA4-26B is a murine antibody that when conjugated (using, for example, reaction conditions described above in Example 1) to calicheamicin produces a large amount of aggregate with low calicheamicin loadings of 1 M/M or less on monomeric conjugate, and yields below 50% under all conditions tested. When deglycosylated using the conditions described above in Example 2 and conjugated with the AcBut derivative, the monomeric conjugate had a loading almost double that obtained with the native antibody and had a yield of greater than 90% with no appreciable aggregate being formed during the reaction or present in the purified material.

### Example 10

Another murine antibody, MOPC (a IgG1-k mouse antibody with unknown specificity that is commonly used as negative control in immunodetection methods), when conjugated (using conditions described above in Example 1, for example) to the AcBut derivative had very poor results of less than 30% yield of monomeric conjugate with a calicheamicin loading of approximately 1 M/M antibody. When deglycosylated using the conditions described above in Example 2, this antibody resulted in a conjugate with a calicheamicin loading of 1-2 M/M with greater than 50% yield.

### Example 11

The murine antibody M198 shows one of the worst conjugation profiles with the AcBut derivative. Reaction with addition of 5% by weight of the AcBut derivative of calicheamicin resulted in over 50% aggregate containing almost 100% of the added drug when conjugated using standard conditions such as those found above in Example 1, for example. Deglycosylation using the conditions described above in Example 2 results in a marginal improvement, with only 85% of the added drug contained in the aggregate fraction.

### Example 12

Incorporation of calicheamicin into conjugate using the AcB ut derivative with hP67.6 antibody results in approximately 50% of the initial amount in the reaction mixture incorporated into the conjugate. For example, addition of 2% activated derivative resulted in a monomeric conjugate yield of 80-90% with a loading of 1-1.5 M/M; addition of 4% activated derivative resulted in a 75-85% yield with a loading of 2-3 M/M; and addition of 6% activated derivative resulted in a 70-80% yield of 3-4 M/M. Higher percentages of added derivative give the same 3-4 M/M loading as with 6% addition with calicheamicin going to aggregate instead of monomeric conjugate. The remaining activated calicheamicin derivative is taken up in aggregated protein.

When enzymatically deglycosylated, hP67.6 conjugates well, there is almost 100% incorporation of the activated calicheamicin derivative into non-aggregated monomeric conjugate. Similar incorporation values for the deglycosylated form have been seen with all other antibodies investigated, such as Herceptin, for example.

### Example 13

Conjugation (using typical conditions such as those, for example, found above in Example 1) of the cFLK-1 antibody (anti-KDR antibody) with the DM derivative of calicheamicin resulted in a conjugate with approximately 3 M/M loading and a 50% yield of monomer. When deglycosylated using the conditions described in Example 2, this antibody resulted in a monomeric conjugate with a loading of 3 M/M, yet with almost 100% yield due to the lack of aggregate formed.

### Example 14

Antibody 6/13 when conjugated as described above in Example 1, for example, with the AcPAc derivative of calicheamicin resulted in very low loading of monomer due to the formation of greater than 75% aggregate. When deglycosylated using the conditions described above in Example 2, a monomeric conjugate was obtained with a loading of approximately 2 M/M and in a yield of greater than 50%.

### Example 15

In the case of the 1 B10 antibody (anti-human proteinase PR3 antibody), a conjugation (using, for example, conditions described above in Example 1) with a 5% by weight addition of the AcPAc derivative of calicheamicin resulted in more than 50% aggregate in the reaction mixture. Using deglycosylated 1B10 (deglycosylated using the conditions described in Example 2), a higher percent by weight addition of 8% was used of the AcBut derivative. Typically, the AcBut derivative and the higher percent calicheamicin added to the reaction should both lead to poorer conjugation; however, they in fact resulted in a reaction with only approximately 20% aggregate and a final monomeric conjugate loading of approximately 3 M/M.

### Example 16

Conjugation of both the AcBut and DM derivatives of calicheamicin to the G544 antibody (anti-CD22 antibody) was improved by deglycosylation using the conditions described in Example 2. For the DM derivative, deglycosylation of the antibody resulted in almost 100% incorporation of calicheamicin and yield of monomeric conjugate with a loading of 4 M/M when conjugated using, for example, the conditions described above in Example 1. By contrast the unmodified antibody under similar conditions with the DM derivative resulted in a loading of 2-3 M/M and incorporation and yield of 75-80%.

### Example 17

Antibody 225 (murine anti-EGFR antibody) conjugated (using, for example, the conditions described above in Example 1) to the DM derivative of calicheamicin resulted in a conjugate with a loading of 2 M/M and a yield of approximately 75% while the deglycosylated version (using the conditions described above in Example 2) resulted in a conjugate with a loading of 3 M/M and yield of approximately 85%.

### Example 18

Under the best conditions found, unmodified H8 antibody (anti-5T4 antibody) when conjugated to the AcBut derivative of calicheamicin resulted in a yield between 30-50% and a loading of (at best) 1 M/M of unaggregated antibody. With the deglycosylated form (using the conditions described above in Example 2), it was possible to obtain a yield of over 50% monomeric conjugate with a loading of approximately 2-3 M/M, even without optimization of reaction conditions.

### Example 19

While the unmodified hS193 antibody (anti-Lewis Y antibody) showed high loading and yield when conjugated to the AcBut derivative of calicheamicin using, for example, typical reaction conditions such as those describe above in Example 2, it did exhibit slight improvement in the amount of aggregate formed during the reaction as shown in the size exclusion purification chromatographs of conjugations done under similar conditions.

Conjugation of two native versions of the hS193 antibody as compared to four genetically engineered versions (G193-NS, G193-NK, G193-NQ, G193-ND) lacking the normal sites for glycosylation had the following results in Table 2, which confirm the role of deglycosylation in conjugation improvement. Under conditions that give comparable aggregate levels in monomeric conjugate, the percent of unconjugated protein is significantly higher (avg. 39.8) in the native glycosylated forms (both the IgG1 or IgG4 antibody) as compared to the percent unconjugated (avg. 5.81) in the aglycosyl versions.

TABLE 2

| mAb | Percent Aggregate | Percent Unconjugated Protein |
|---|---|---|
| G193-NS | 1.53 | 1.84 |
| G193-NK | 4.88 | 9.37 |
| G193-NQ | 5.33 | 5.85 |
| G193-ND | 5.93 | 6.16 |
| G193-IgG4 | 4.48 | 38.7 |
| G193-IgG1 | 5.68 | 39.7 |
| Native average | 5.80 | 39.8 |
| Aglycosyl average | 4.42 | 5.81 |

### Example 20

To confirm the role of carbohydrate in the conjugation reaction, separation by affinity chromatography using Concanavalin A (Con A) Sephadex (Amersham Biosciences) was performed in the following manner. Each antibody solution was passed over a ConA column equilibrated with 0.1M sodium acetate buffer pH6.0 containing calcium, magnesium, and manganese cations needed for saccharide binding to the concanavalin lectin. The flow through fraction of the antibody solution, which did not bind, was collected as fraction 1. Since this column binds antibody based on the presence of specific saccharides, the unbound fraction is that material with less oligosaccharide attached. Antibody bound to the column was eluted as either fraction 2 which came off as a well defined peak using a high molarity pyranoside solution as eluent, or as fraction 3 which represents tightly bound material that eluted slowly as a tailing fraction. The difference in these fractions is due to differences in the amount and type of oligosaccharide present.

Results for various antibodies are listed below in Table 3. Comparison of the conjugation properties of these antibodies with their ConA separation profiles show a correlation of poor conjugation (using typical reaction conditions such as those described above in Example 1) with increased bound fractions 2 and 3. The worst conjugation behavior leading to substantial aggregation was shown by antibodies. M198 and CB006 which also had the most material in their tightly bound fractions. Antibodies such as Herceptin that have less fraction 3 had better conjugation properties than the above mentioned antibodies, as did g544 which despite having more lightly bound material had very little tightly bound.

TABLE 3

| Antibody | Fraction 1 | Fraction 2 | Fraction 3 |
|---|---|---|---|
| hP67.6 | 50% | 25% | 25% |
| BR96 | 43% | 30% | 27% |
| CB006 | 25% | 40% | 35% |
| Herceptin | 60% | 25% | 15% |
| M198 | 10% | 50% | 40% |
| g544 | 25% | 70% | 5% |
| m544 | 10% | 50% | 40% |
| BC8 | 40% | 50% | 10% |
| hS193 | 80% | 15% | 5% |
| Deglyco-hP67.6 | 90% | 5% | Less than 5% |

Using the ConA separated fractions that are described above and performing conjugation reactions with these fractions had the following results. Fraction 1 of hP67.6 when conjugated with 8% by weight of the AcBut calicheamicin derivative resulted in a monomeric conjugate with a loading of 7 M/M and approximately 90% yield. The deglycosylated version of hP67.6, of which 90% is unbound on ConA, resulted in the same conjugation pattern of high loading and yield with no aggregate. In comparison, the unseparated antibody under these reaction conditions resulted in a loading of 3-4 M/M with substantial aggregation leading to a yield of less than 40%. Conjugation of fraction 2 under the normal reaction conditions of 4% calicheamicin addition resulted in a monomeric conjugate with only 1 M/M loading as compared to the 3-4 seen with the unseparated antibody. The Ml 98 antibody as seen previously cannot be readily conjugated without almost complete aggregation occurring. Using the ConA fraction 1 of this antibody resulted in a conjugate with a calicheamicin loading of approximately 2 M/M in a yield of 50%.

These results indicate that from the same batch of antibody, the material having more oligosaccharide present conjugates poorly, while the antibody having less oligosaccharide conjugates better. Antibody hS193, which had a ConA profile closest to that of deglycosylated antibody, was capable of high conjugate loadings in high yield similar to the deglycosylated forms. Even so, when deglycosylated, this antibody also exhibited some improvement in conjugation, as shown in Example 19. In addition the different conjugation characteristics of the 5/44 antibody in either its murine (m544) or CDR grafted (G544) form reflect the differences in their oligosaccharide profiles as indicated by their ConA binding.

### Example 21

Cation exchange chromatography was used to show the pattern of calicheamicin conjugation for several antibodies and to show the difference in loading distribution for unmodified and deglycosylated antibody. Upon maximum loading conditions for the unmodified hP67.6 antibody, the cation exchange pattern shows two distinct subpopulations of protein that differentially incorporate calicheamicin either poorly or well. This uneven distribution leads to a higher than desired, or expected by statistical distribution predictions, level of unconjugated antibody. When deglycosylated antibody is used, an even distribution of calicheamicin loading is accomplished, which resulted in less unconjugated antibody and a more homogenous conjugate.

### Example 22

A study of reaction kinetics for the hP67.6 AcBut conjugation showed that a reaction of deglycosylated antibody purified after reacting for 1 min. resulted in a loading of almost 6 M/M, while the unmodified antibody reacted under identical conditions achieved less than a 1 M/M loading in this time.

### Example 23

In contrast to normally glycosylated proteins, such as the antibodies mentioned in previous examples, conjugations of the naturally unglycosylated protein human serum albumin (HSA) does not show an improvement upon treatment with deglycosylation enzymes.

### Example 24

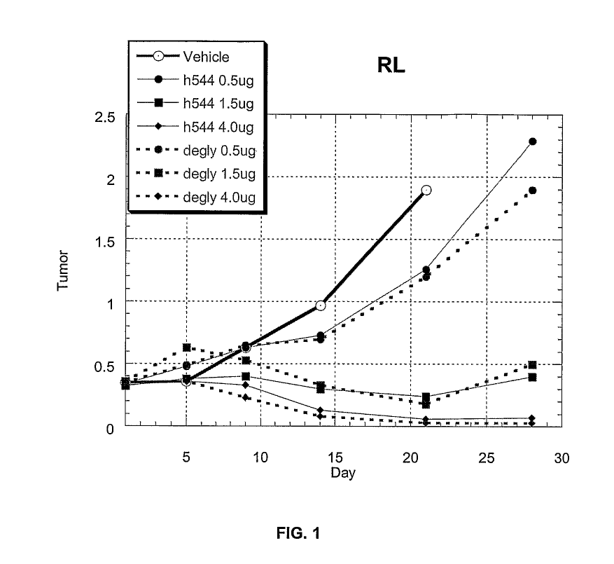

To show that conjugates made using a deglycosylated antibody have the same activity as the native species conjugates, a xenograft experiment was performed using both forms. In FIG. 1, the results of xenografts of the RL cell line into nude mice shows that the results of the conjugates of the humanized 544 antibody given at identical doses had the same efficacy in preventing tumor growth whether the antibody was native or enzymatically deglycosylated as described in Example 2.

A second concern for modified proteins is that the pharmacokinetic properties of the altered species should not be deleteriously affected. To show this conjugates of the humanized P67.6 antibody were injected into rats and the pharmacokinetic properties of the two species were compared. Plasma half-life and blood clearance values for the two conjugates indicated that there was no appreciable difference between the two.

What is claimed is:

1. A process for preparing a calicheamicin conjugate comprising removing glycosylation of a protein and reacting (i) an activated calicheamicin—hydrolyzable linker derivative and (ii) the deglycosylated protein.

2. The process of claim 1, wherein the protein is an antibody.

3. The process of claim 2, wherein the antibody is an anti-CD33 antibody, an anti-CD22 antibody, an anti-Lewis Y antibody, an anti-5T4 antibody or an anti-CD20 antibody.

4. The process of claim 3, wherein the antibody is hp67.6, G544, G193, H8, or rituximab.

5. The process of claim 1 or 2, wherein deglycosylation is accomplished by stepwise monosaccharide removal.

6. The process of any of claims 1-5, wherein the calicheamicin derivative is an N-acyl derivative of calicheamicin or a disulfide analog of calicheamicin.

7. The process of claim 6, wherein the calicheamicin derivative is N-acetyl gamma calicheamicin dimethyl hydrazide (N-acetyl calicheamicin DMH).

8. The process of any of claims 1-7, wherein the hydrolyzable linker is 4-(4-acetylephenoxy) butanoic acid (AcBut) or (3-Acetylphenyl) acetic acid (AcPAc).

9. The process of any of claims 1-8, wherein the process further comprises purifying the calicheamicin conjugate.

10. A calicheamicin conjugate produced by the process of any of claims 1-9.
